(19) United States
(12) Patent Application Publication
(10) Pub. No.: US 2013/0071530 A1
(43) Pub. Date: Mar. 21, 2013

(54) CASHEW NUT SHELL LIQUID HAVING IMPROVED STABILITY

(71) Applicants: Shinji Ito, Sodegaura-shi (JP); Kyo Nagashima, Sodegaura-shi (JP); Masami Mochizuki, Sodegaura-shi (JP); Seika Ooiwa, Sodegaura-shi (JP)

(72) Inventors: Shinji Ito, Sodegaura-shi (JP); Kyo Nagashima, Sodegaura-shi (JP); Masami Mochizuki, Sodegaura-shi (JP); Seika Ooiwa, Sodegaura-shi (JP)

(73) Assignee: IDEMITSU KOSAN CO., LTD., Tokyo (JP)

(21) Appl. No.: 13/701,596

(22) PCT Filed: Jun. 3, 2011
(86) PCT No.: PCT/JP2011/062829

§ 371 Date: Dec. 3, 2012

(30) Foreign Application Priority Data
Jun. 3, 2010 (JP) 2010 128201

(51) Int. Cl. A23K 3/00; C07C 65/05; A23K 1/00

(52) U.S. Cl. CPC A23K 3/00; A23K 1/001; C07C 65/05
USPC 426/330; 426/655; 562/475

(57) ABSTRACT

The present invention provides a composition comprising one or more acids and a non-heated cashew nut shell liquid, wherein decarboxylation of an anacardic acid in the non-heated cashew nut is inhibited by the one or more acids.

### TECHNICAL FIELD

The present invention relates to a composition containing an acid and a non-heated cashew nut shell liquid (CNSL), in which a decarboxylation of an anacardic acid in the non-heated cashew nut is inhibited by the acid, and a non-heated cashew nut shell liquid preparation and a feed each using the composition. The present invention also relates to a method of inhibiting a decarboxylation reaction of an anacardic acid in a non-heated cashew nut shell liquid, comprising adding an acid to the non-heated cashew nut shell liquid.

### BACKGROUND ART

A cashew nut shell liquid is an oily liquid contained in a shell of a fruit of a cashew nut tree (Anacardium occidentale L.). The cashew nut shell liquid mainly contains, as its components, an anacardic acid, cardanol, cardol, and methylcardol.

A method of preparing a cashew nut shell liquid includes a heating method and a solvent-extraction method, but in general, the cashew nut shell liquid is used after converting the anacardic acid into a cardanol by heat treatment at a production district of the cashew nut.

This is because the anacardic acid that is a component of the non-heated cashew nut shell liquid easily decarboxylates and may form a foam during transportation. Further, the non-heated cashew nut shell liquid solidifies at about 20° C. and loses its fluidity, and hence it is difficult to transport the liquid. For that reason, the transportation of the non-heated cashew nut shell liquid is largely constrained, which prevents further industrial uses.

Patent Documents 1 to 3 describe examples of applications for industrial products, but all the cashew nut shell liquids used are heat-treated products.

Patent Documents 4 to 6 mention the applications of the non-heated cashew nut shell liquid and the anacardic acid as a component of the liquid for feed. Because of the above-mentioned restrictions of stability and transportation, it is difficult to realize the applications including solving physical and economical problems. Further, Patent Documents 4 to 6 describe the applications of the non-heated cashew nut shell liquid, but there are no findings which focus on improvement in its stability and handling and seek to improve the stability and handling.

Accordingly, it has been desired to develop a method of stabilizing the non-heated cashew nut shell liquid so that the liquid can easily be transported.

Patent Document 7 describes a method of stabilizing an anacardic acid by adding an alkaline to a cashew nut shell liquid containing the anacardic acid and mixing the resultant. However, the document 7 does not describe the stabilization of the anacardic acid by adding an acid to the cashew nut shell liquid. Further, the document 7 does not describe the solidification of the non-heated cashew nut shell liquid at about 20° C. can be prevented by blending a carrier in the non-heated cashew nut shell liquid supplemented with an acid.

### PRIOR ART DOCUMENTS

### Patent Documents

Patent Document 1: JP 2008-144171 A
Patent Document 2: JP 2006-111839 A
Patent Document 3: JP 2003-252893 A
Patent Document 4: JP 2003-238400 A
Patent Document 5: JP 2001-151675 A
Patent Document 6: JP 8-231410 A
Patent Document 7: JP 2010-88363 A

### SUMMARY OF THE INVENTION

An object of the present invention is to enable easy transportation of a non-heated cashew nut shell liquid, in particular, an anacardic acid as a component thereof without decarboxylation. Another object of the present invention is to enable easy transportation of a non-heated cashew nut shell liquid that solidifies at about 20° C. even at a low temperature.

The inventors of the present invention have conducted intensive studies to solve the above-mentioned problems, and as a result, the inventors have found that the addition of an acid to the non-heated, cashew nut shell liquid can inhibit the decarboxylation of an anacardic acid.

Further, the inventors of the present invention have found that the blending of a carrier such as a grain powder or silica with the non-heated cashew nut shell liquid having the decarboxylation inhibited through the addition of the acid enables the transportation of the non-heated cashew nut shell liquid without solidification even at a low temperature of about 20° C. or less.

Thus, the inventors of the present invention have completed the present invention.

That is, the summary of the present invention is as follows.

(1) A composition comprising one or more acids and a non-heated cashew nut shell liquid, wherein decarboxylation of an anacardic acid in the non-heated cashew nut is inhibited by the one or more acids.

(2) The composition according to (1), wherein the acid has a pKa of 4.5 or less.

(3) The composition according to (1) or (2), wherein an amount of the acid is 0.5 wt % or more with respect to the non-heated cashew nut shell liquid.

(4) A non-heated cashew nut shell liquid preparation, comprising the composition according to any one of (1) to (3) and one or more carriers.

(5) The non-heated cashew nut shell liquid preparation according to (4), wherein the carrier is silica.

(6) A feed comprising a non-heated cashew nut shell liquid having an inhibited decarboxylation of an anacardic acid, which is produced by adding the composition according to any one of (1) to (3) and/or the non-heated cashew nut shell liquid preparation according to (4) or (5).

(7) A method of inhibiting a decarboxylation reaction of an anacardic acid in a non-heated cashew nut shell liquid, comprising adding one or more acids to the non-heated cashew nut shell liquid.

(8) A method of producing a non-heated cashew nut shell liquid preparation according to (4) or (5), comprising mixing one or more acid components with one or more carriers and further mixing the carrier mixed with the acid component with the non-heated cashew nut shell liquid.

(9) A method of inhibiting a decarboxylation reaction of an anacardic acid, comprising adding one or more acids to the anacardic acid.

The addition of an acid to a non-heated cashew nut shell liquid can inhibit the decarboxylation of an anacardic acid that is a component of the non-heated cashew nut shell liquid. Further, the blending of the carrier with the non-heated cashew nut shell liquid supplemented with the acid can prevent the solidification at about 20° C. Accordingly, the non-heated cashew nut shell liquid can be easily used for industrial applications.

### BRIEF DESCRIPTION OF THE DRAWINGS

FIG. 1 shows inhibition of decarboxylation reactions of non-heated cashew nut shell liquids in the cases of using 1 wt % various acids. The bar graphs show, in a descending order, cardanols 15:1, 15:2, and 15:3, and anacardic acids 15:1, 15:2, and 15:3.

FIG. 2 shows the inhibition of the decarboxylation reactions of the non-heated cashew nut shell liquids in the cases of using 10 wt % various acids. The bar graphs show, in a descending order, cardanols 15:1, 15:2, and 15:3, and anacardic acids 15:1, 15:2, and 15:3.

FIG. 3 shows the inhibition of the decarboxylation reactions of the non-heated cashew nut shell liquids in the cases of using various concentrations of sulfuric acids. The bar graphs in FIG. 3A show, in a descending order, cardanol 15:1 and anacardic acid 15:1. The bar graphs in FIG. 3B show, in a descending order, cardanol 15:3 and anacardic acid 15:3.

FIG. 4 shows the inhibition of the decarboxylation reactions in the non-heated cashew nut shell liquid preparations in the case of using a 10 wt % phosphoric acid (acid was preliminarily added to non-heated cashew nut shell liquid). The bar graphs show, in a descending order, cardanol 15:3 and anacardic acid 15:3.

FIG. 5 shows the inhibition of the decarboxylation reactions in the non-heated cashew nut shell liquid preparations in the case of using the 10 wt % phosphoric acid (acid was added to carriers in advance). The upper bar graphs show, in a descending order, cardanol 15:1 and anacardic acid 15:1, and the lower bar graphs show, in a descending order, cardanol 15:3 and anacardic acid 15:3.

FIG. 6 shows the inhibition of the decarboxylation reactions in feeds in the case of using the 10 wt % phosphoric acid. The bar graphs show, in a descending order, cardanol 15:3 and anacardic acid 15:3.

### MODE FOR CARRYING OUT THE INVENTION

The composition of the present invention contains one or more acids and a non-heated cashew nut shell liquid and is characterized in that a decarboxylation of an anacardic acid in the non-heated cashew nut is inhibited by the one or more acids.

The cashew nut shell liquid to be used in the present invention is an oily liquid contained in the shell of the seed of a cashew nut tree (Anacardium occidentale L.). The cashew nut shell liquid contains, as components thereof, anacardic acid, cardanol, cardol, and methylcardol.

Non-heated cashew nut shell liquid (hereinafter, referred to as a cashew nut shell liquid) extracted by compressing the shell of a cashew nut contains 55 to 80 mass % anacardic acid, 5 to 20 mass % cardanol, and 5 to 30 mass % cardol as described in J. Agric. Food Chem. 2001, 49, 2548-2551.

It should be noted that there are three kinds of anacardic acids: an anacardic acid having three double bonds at 8-, 11-, and 14-positions (hereinafter, referred to as anacardic acid 15:3); an anacardic acid having two double bonds at 8- and 11-positions (hereinafter, referred to as anacardic acid 15:2); and an anacardic acid having one double bond at 8-position (hereinafter, referred to as anacardic acid 15:1).

The present invention further includes a method of inhibiting a decarboxylation reaction of an anacardic acid, which is characterized by adding one or more acids to the anacardic acid.

The cashew nut shell liquid to be used in the present invention can be obtained as a vegetable oil extracted by compressing the shell of a cashew nut. Further, the cashew nut shell liquid can also be obtained by extracting, e.g., solvent-extracting a cashew nut shell. In addition, the cashew nut shell liquid can be obtained according to a method described in JP 08-231410 A, e.g., by a solvent extraction method.

The cashew nut shell liquid may also be a liquid obtained by pulverizing/crushing the shell of a cashew nut.

Further, a commercially available cashew nut shell liquid product may also be used.

The acid to be used in the present invention has a pKa of preferably 4.5 or less, more preferably 4.0 or less. Specific examples of the acids include, but are not limited thereto, a formic acid, a malic acid, a citric acid, a succinic acid, a tartaric acid, a phosphoric acid, a nitric acid, a hydrochloric acid, an oxalic acid, a malonic acid, a glutaric acid, an adipic acid, a fumaric acid, a maleic acid, a phthalic acid, an isophthalic acid, a phytic acid, a sulfuric acid, a terephthalic acid, a lactic acid, a sulfamic acid, a hydroxyacetic acid, a phosphonic acid, and a gluconic acid. In the present invention, the pKa value refers to a value calculated at 25° C. when water is used as a solvent.

The amount of the acid to be used is preferably 0.5 wt % or more, more preferably 1 to 20 wt % with respect to the cashew nut shell liquid (CNSL). It should be noted that, in the case where the pKa of the acid is higher than 2, it is necessary to increase the acid to be added. For example, the amount may be adjusted to about 5 to 10 wt %.

The composition of the present invention can be produced by adding one or more acids to the non-heated cashew nut.

The non-heated cashew nut shell liquid preparation of the present invention is preferably a preparation containing the composition of the present invention and one or more carriers. For example, the composition of the present invention may be mixed with the one or more carriers to produce the non-heated cashew nut shell liquid preparation.

Examples of an inorganic carrier include, but are not limited thereto, a silicic acid and a salt thereof (such as silica), vermiculite, diatomaceous earth, talc, kaolin, and bentonite.

In the cases where a silicic acid and a salt thereof are used as the inorganic carriers, the carriers preferably each have a specific surface area of 500 m³/g or less so as not to cause significant oxidation reactions. In the case of using silica as the inorganic carrier, the blending ratio (weight ratio) is preferably silica/non-heated cashew nut shell liquid=1/3.0 to 1/0.1. In the case of using another inorganic carrier, the same specific surface area and blending ratio as described above may be employed. It should be noted that the specific surface area of silica can be measured by a BET method.

It should be noted that grain powders or the below-mentioned feed component such as corn grain, corn powder, milo, soybean cake, oat, wheat flour short, wheat coarse flour, alfalfa, clover, defatted rice bran, white fishmeal, fish meal, yeast, molasses, meat pieces, or born meal may be used as an organic carrier.

The non-heated cashew nut shell liquid preparation of the present invention is not particularly limited as long as the preparation contains a non-heated cashew nut shell liquid, one or more carriers, and one or more acid components. Therefore, the preparation may be produced by: preliminarily mixing the acid component with the carrier; and further mixing the carrier mixed with the acid component with the non-heated cashew nut shell liquid. The effect of the acid component in the carrier can inhibit the decarboxylation of the non-heated cashew nut shell liquid in a state in which the acid is mixed with the carrier.

The non-heated cashew nut shell liquid preparation of the present invention may include one or more antioxidants in addition to a non-heated cashew nut shell liquid, a carrier, and an acid component. Examples thereof include ethoxyquin, t-butylhydroxytoluene, t-butylhydroxyanisole, t-butylhydroquinone, an ascorbic acid and esters thereof, Vintamin E, a gallic acid and esters thereof, an erythorbic acid, a chlorogenic acid, sulfite, thiosulfate, phosphite, hypophosphite, and phosphate.

The non-heated cashew nut shell liquid preparation of the present invention may include one or more antibiotics in addition to a non-heated cashew nut shell liquid, a carrier, and an acid component. Examples thereof include zinc bacitracin, avilamycin, alkyl trimethyl anmonium calcium tetracycline, efrotomycin, enramaycin, chlortetracycline, sedecamycin, Semduramicin, narasin, nosiheptide, virginiamycin, bicozamycin, flavophospholipol, polynactin, monensin, salinomycin, lasalocid, lysoceline, lonomycin, avoparcin, colistin sulfate, tylosin phosphate, amprolium-ethopabate, sulfaquinoxaline, morantel citrate, decoquinate, nicarbazin, halofuginone polystyrene-sulfonate, kitasamycin, tiopeptone, Destomycin A, Hygromycin B, and salts thereof.

The non-heated cashew nut shell liquid preparation of the present invention may be formulated into a powder formulation by containing a carrier such as grain powders or silica. That is, the non-heated cashew nut shell liquid preparation of the present invention can be produced by mixing the non-heated cashew nut shell liquid, the one or more carriers, the one or more acid components, and if necessary, one or more optional components and formulating the mixture into a powder formulation. Such powder formulation of the present invention can be used as a feed without mixing the formulation with another optional component.

The non-heated cashew nut shell liquid preparation of the present invention can be formulated into not only a powder formulation but also a granular formulation such as a pellet. In this case, not only the inorganic carrier and the acid component but also one or more hardened oils may be added to the non-heated cashew nut shell liquid. As the hardened oil, an oil obtained by hardening a palm oil, a soybean oil, a rapeseed oil, or the like is used. The melting point of the hardened oil is preferably 45 to 65° C. It should be noted that a general extruding granulator can be used for producing the preparation as a pellet.

The non-heated cashew nut shell liquid preparation of the present invention may be coated. For example, after granulation, the preparation can be coated with one or more coating agents selected from zein, shellac, hydroxypropylmethylcellulose (HPMC), pullulan, hemilose, glucose, lactose, trehalose, and starch. Alternatively, the preparation may be coated with a sheet containing the coating agents as components.

The present invention includes a feed containing the composition and/or the non-heated cashew nut shell liquid preparation of the present invention. In the present invention, the non-heated cashew nut shell liquid to be contained in the feed has the decarboxylation inhibited by the one or more acid components.

In the feed of the present invention, the kind and blending ratio of the feed component to be blended with the composition and/or the cashew nut shell liquid preparation of the present invention are not particularly limited. The feed may be one conventionally given to animals. For example, the feed may be prepared using one or more feed components such as corn grain, corn powder, milo, soybean cake, oat, wheat flour short, wheat coarse flour, alfalfa, clover, defatted rice bran, white fish meal, fish meal, yeast, molasses, meat pieces, born meal, calcium carbonate, dibasic calcium phosphate, yellow grease, vitamins, or minerals.

The feed of the present invention can be produced by adding the cashew nut shell liquid preparation as it is to a feed component and mixing the resultant. On this occasion, when a powdery or solid cashew nut shell liquid preparation is used, the form of the cashew nut shell liquid preparation may be modified into a liquid form or a gel form for the purpose of facilitating the mixing process. In this case, one or more of the followings may be used as a liquid carrier: water; a vegetable oil such as soybean oil, rapeseed oil, or corn oil; liquid animal oil; and a water-soluble polymer compound such as polyvinyl alcohol, polyvinylpyrrolidone, or polyacrylic acid. Further, in order to keep the uniformity of the cashew nut shell liquid in the feed, the feed preferably contains one or more of alginic acid, sodium alginate, a xanthan gum, casein sodium, an arabic rubber, a guar gum, and a water-soluble polysaccharide such as tamarind seed polysaccharide.

The feed of the present invention is suitable for breeding livestock such as cows, pigs, chickens, sheep, horses, and goats. The amount of feed ingested by an animal may be appropriately adjusted depending on the animal's species, body weight, age, sex, health condition, feed component, etc. In this case, the amount of cashew nut shell liquid contained in the feed is preferably 0.005 to 500 g per animal per day, more preferably 0.05 to 100 g per animal per day.

Any method usually used may be adopted as a method of feeding animals and a method of raising animals depending on the species of animals.

### EXAMPLES

### Example 1

### Sample preparation

500 kg of cashew nut shells were purchased from Cashew Trading Co., Ltd., and the shells were compressed, thereby producing 158 kg of a cashew nut shell liquid (non-heated cashew nut shell liquid).

Further, the following 2 grade silica were used as the silica to be used as the carrier.

Sipernat 22 (pH 6.5) (manufactured by Evonik Degussa Japan Co., Ltd.)
Carplex #1120 (pH 10.6) (manufactured by Evonik Degussa Japan Co., Ltd.)

The pKa of the acid used is as follows.

Sulfuric acid (−3)
Nitric acid (−1.6)
Phosphoric acid (2.1)
Formic acid (3.8)
Acetic acid (4.8)

### Example 2

### HPLC Measurement

As devices, HPLC (Waters 600, Nihon Waters K.K.), a detector (Waters 490E, Nihon Waters K.K.), a printer (Chromatopak C-R6A, Shimadzu Corporation), and a column (SUPELCOSIL LC18, SUPELCO, Inc.) were used. Measurement was performed under the following conditions: solvent acetonitrile:water:acetic acid=80:20:1 (volume ratio); flow rate 2 ml/ml; temperature 25° C.; absorbance 280 nm.

pH Measurement

The pH of silica was measured according to the following procedure.

A 5 wt % silica-water suspension was prepared using ion-exchanged water, and the electrical potential was measured using glass electrodes to determine the pH.

### Example 3

### Inhibition of Decarboxylation Reaction in Cases of Addition of 1 wt % or 10 wt % Various Acids

10 g of a non-heated cashew nut shell liquid were taken in each beaker, and a 1 wt % sulfuric acid, a 1 wt % nitric acid, 1 wt % and 10 wt % phosphoric acids, 1 wt % and 10 wt % formic acids, and 1 wt % and 10 wt % acetic acids were added thereto, respectively, followed by stirring to homogenize each mixture.

The respective beakers were sealed and then left to stand in a thermostat chamber at 80° C., and 4 days later, samples were collected and subjected to composition analyses by HPLC.

An anacardic acid (AA) is converted into a cardanol (CD) by a decarboxylation reaction, and hence Tables 1 and 2 describe the compositions of anacardic acid, cardanol, and cardol before and after the beakers were left to stand in the thermostat chamber. Depending on the number of unsaturated bonds in a fat chain which binds to an aromatic ring and has 15 carbon atoms, there are mainly the following three types of each of anacardic acid, cardanol, and cardol (anacardic acid; AA15:1, AA15:2, and AA15:3, cardanol; CD15:1, CD15:2, and CD15:3, cardol; 15:1, 15:2, and 15:3).

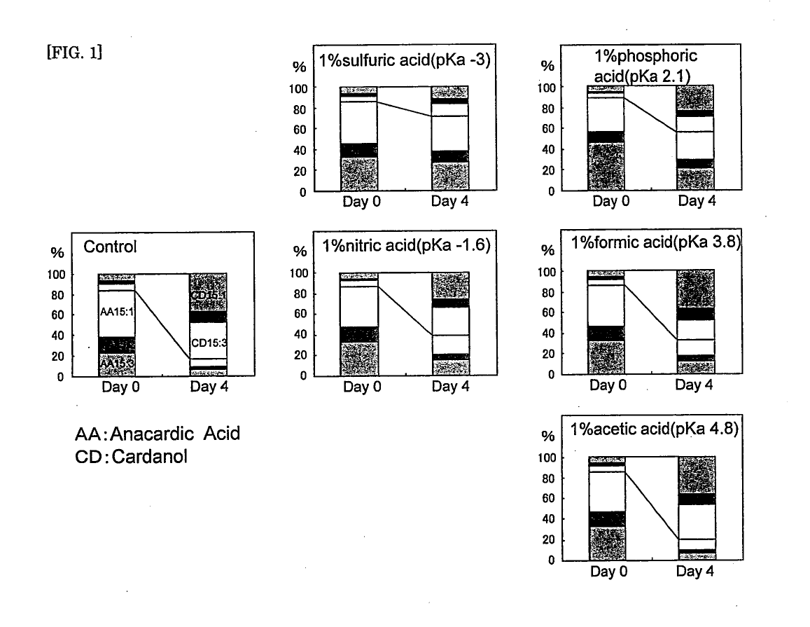

In the cases where each acid was added in an amount of 1 wt % (FIG. 1 and Table 1), the ratios of the decarboxylation (anacardic acid→cardanol) were found to decrease 4 days later depending on the levels of the pKa. In the case of an acetic acid having a pKa of 4.8, the decarboxylation ratio after the addition of the 1 wt % acetic acid was almost the same as that in the case of the addition of no acid (control).

Note that cardol is listed on Table 1, but not in FIG. 1.

TABLE 1

| | | | Day 0 | | Day 4 |
|---|---|---|---|---|---|
| | | Day 0 | (%) | Day 4 | (%) |
| Control | Anacardic acid C15:3 | 1878 | 16.9 | 753 | 5.4 |
| | Anacardic acid C15:2 | 1335 | 12.0 | 311 | 2.2 |
| | Anacardic acid C15:1 | 3849 | 34.5 | 955 | 6.8 |
| | cardanol C15:3 | 533 | 4.8 | 4213 | 30.0 |
| | cardanol C15:2 | 213 | 1.9 | 1320 | 9.4 |
| | cardanol C15:1 | 591 | 5.3 | 4281 | 30.5 |
| | cardol C15:3 | 1784 | 16.0 | 1700 | 12.1 |
| | cardol C15:2 | 542 | 4.9 | 524 | 3.7 |
| | cardol C15:1 | 422 | 3.8 | 0 | 0.0 |
| | Total | 11147 | 100.0 | 14058 | 100.0 |
| 1% | Anacardic acid C15:3 | 2865 | 25.7 | 2600 | 22.4 |
| sulfuric | Anacardic acid C15:2 | 1140 | 10.2 | 1099 | 9.5 |
| acid | Anacardic acid C15:1 | 3522 | 31.7 | 3176 | 27.4 |
| | cardanol C15:3 | 453 | 4.1 | 1119 | 9.6 |
| | cardanol C15:2 | 200 | 1.8 | 396 | 3.4 |
| | cardanol C15:1 | 530 | 4.8 | 1214 | 10.5 |
| | cardol C15:3 | 1601 | 14.4 | 1507 | 13.0 |
| | cardol C15:2 | 451 | 4.1 | 483 | 4.2 |
| | cardol C15:1 | 365 | 3.3 | 0 | 0.0 |
| | Total | 11126 | 100.0 | 11594 | 100.0 |
| 1% nitric | Anacardic acid C15:3 | 3485 | 25.8 | 1743 | 12.5 |
| acid | Anacardic acid C15:2 | 1464 | 10.9 | 753 | 5.4 |
| | Anacardic acid C15:1 | 4176 | 31.0 | 2142 | 15.3 |
| | cardanol C15:3 | 578 | 4.3 | 3149 | 22.5 |
| | cardanol C15:2 | 239 | 1.8 | 976 | 7.0 |
| | cardanol C15:1 | 628 | 4.7 | 3097 | 22.2 |
| | cardol C15:3 | 1873 | 13.9 | 1607 | 11.5 |
| | cardol C15:2 | 581 | 4.3 | 509 | 3.6 |
| | cardol C15:1 | 458 | 3.4 | 0 | 0.0 |
| | Total | 13483 | 100.0 | 13976 | 100.0 |
| 1% | Anacardic acid C15:3 | 5077 | 37.1 | 2530 | 17.5 |
| phos- | Anacardic acid C15:2 | 1189 | 8.7 | 1075 | 7.5 |
| phoric | Anacardic acid C15:1 | 3633 | 26.6 | 3143 | 21.8 |
| acid | cardanol C15:3 | 485 | 3.5 | 1871 | 13.0 |
| | cardanol C15:2 | 199 | 1.5 | 565 | 3.9 |
| | cardanol C15:1 | 550 | 4.0 | 2994 | 20.8 |
| | cardol C15:3 | 1699 | 12.4 | 1722 | 11.9 |
| | cardol C15:2 | 477 | 3.5 | 521 | 3.6 |
| | cardol C15:1 | 373 | 2.7 | 0 | 0.0 |
| | Total | 13680 | 100.0 | 14421 | 100.0 |
| 1% formic | Anacardic acid C15:3 | 3003 | 25.7 | 1157 | 10.1 |
| acid | Anacardic acid C15:2 | 1207 | 10.3 | 505 | 4.4 |
| | Anacardic acid C15:1 | 3637 | 31.1 | 1453 | 12.6 |
| | cardanol C15:3 | 503 | 4.3 | 1706 | 14.8 |
| | cardanol C15:2 | 207 | 1.8 | 1096 | 9.5 |
| | cardanol C15:1 | 550 | 4.7 | 3495 | 30.4 |
| | cardol C15:3 | 1687 | 14.4 | 1607 | 14.0 |
| | cardol C15:2 | 512 | 4.4 | 494 | 4.3 |
| | cardol C15:1 | 395 | 3.4 | 0 | 0.0 |
| | Total | 11701 | 100.0 | 11515 | 100.0 |
| 1% acetic | Anacardic acid C15:3 | 3006 | 25.5 | 840 | 6.1 |
| acid | Anacardic acid C15:2 | 1263 | 10.7 | 352 | 2.6 |
| | Anacardic acid C15:1 | 3641 | 30.9 | 1133 | 8.2 |
| | cardanol C15:3 | 518 | 4.4 | 3945 | 28.7 |
| | cardanol C15:2 | 208 | 1.8 | 1250 | 9.1 |
| | cardanol C15:1 | 558 | 4.7 | 4065 | 29.6 |
| | cardol C15:3 | 1687 | 14.3 | 1670 | 12.1 |
| | cardol C15:2 | 515 | 4.4 | 493 | 3.6 |
| | cardol C15:1 | 396 | 3.4 | 0 | 0.0 |
| | Total | 11792 | 100.0 | 13747 | 100.0 |

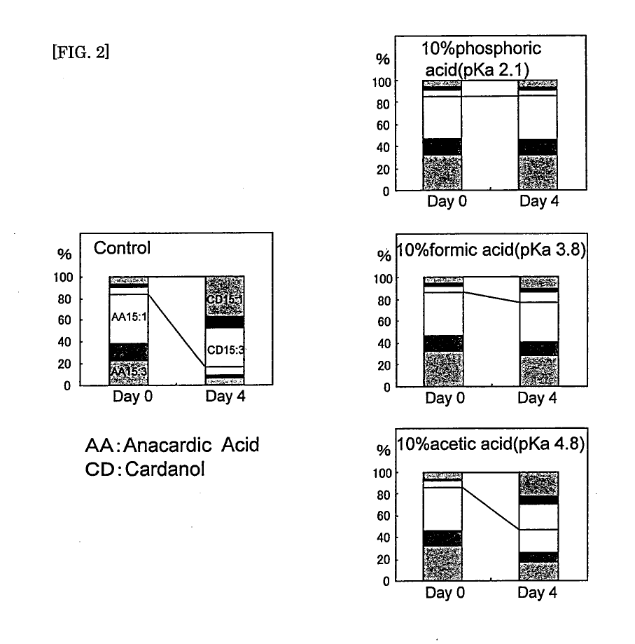

On the other hand, further experiments were performed using the acids each having a relatively low pKa value in an increased addition amount of 10 wt % (FIG. 2 and Table 2), and as a result, the contents of the cardanol components were found to significantly decrease 4 days later up to a formic acid having a pKa of 3.8, and the increases in the addition amounts were found to provide significant effects.

Note that cardol is listed on Table 2, but not in FIG. 2.

TABLE 2

| | | | Day 0 | | Day 4 |
|---|---|---|---|---|---|
| | | Day 0 | (%) | Day 4 | (%) |
| Control | Anacardic acid C15:3 | 1878 | 16.9 | 753 | 5.4 |
| | Anacardic acid C15:2 | 1335 | 12.0 | 311 | 2.2 |
| | Anacardic acid C15:1 | 3849 | 34.5 | 955 | 6.8 |
| | cardanol C15:3 | 533 | 4.8 | 4213 | 30.0 |
| | cardanol C15:2 | 213 | 1.9 | 1320 | 9.4 |
| | cardanol C15:1 | 591 | 5.3 | 4281 | 30.5 |
| | cardol C15:3 | 1784 | 16.0 | 1700 | 12.1 |
| | cardol C15:2 | 542 | 4.9 | 524 | 3.7 |
| | cardol C15:1 | 422 | 3.8 | 0 | 0.0 |
| | Total | 11147 | 100.0 | 14058 | 100.0 |
| 10% | Anacardic acid C15:3 | 2891 | 25.7 | 3277 | 26.0 |
| phos- | Anacardic acid C15:2 | 1218 | 10.8 | 1440 | 11.4 |
| phoric | Anacardic acid C15:1 | 3467 | 30.8 | 3924 | 31.2 |
| acid | Cardanol C15:3 | 474 | 4.2 | 593 | 4.7 |
| | cardanol C15:2 | 185 | 1.6 | 249 | 2.0 |
| | cardanol C15:1 | 516 | 4.6 | 601 | 4.8 |
| | cardol C15:3 | 1614 | 14.4 | 1612 | 12.8 |
| | cardol C15:2 | 489 | 4.4 | 504 | 4.0 |
| | cardol C15:1 | 386 | 3.4 | 381 | 3.0 |
| | Total | 11240 | 100.0 | 12580 | 100.0 |
| 10% | Anacardic acid C15:3 | 3004 | 25.8 | 2376 | 23.5 |
| formic | Anacardic acid C15:2 | 1216 | 10.4 | 990 | 9.8 |
| acid | Anacardic acid C15:1 | 3626 | 31.1 | 2869 | 28.4 |
| | cardanol C15:3 | 503 | 4.3 | 832 | 8.2 |
| | cardanol C15:2 | 207 | 1.8 | 291 | 2.9 |
| | cardanol C15:1 | 541 | 4.6 | 863 | 8.5 |
| | cardol C15:3 | 1679 | 14.4 | 1237 | 12.3 |
| | cardol C15:2 | 475 | 4.1 | 382 | 3.8 |
| | cardol C15:1 | 396 | 3.4 | 257 | 2.5 |
| | Total | 11646 | 100.0 | 10098 | 100.0 |
| 10% | Anacardic acid C15:3 | 2786 | 25.6 | 1872 | 15.0 |
| acetic | Anacardic acid C15:2 | 1114 | 10.2 | 804 | 6.4 |
| acid | Anacardic acid C15:1 | 3384 | 31.1 | 2307 | 18.5 |
| | cardanol C15:3 | 462 | 4.2 | 2435 | 19.5 |
| | cardanol C15:2 | 177 | 1.6 | 741 | 5.9 |
| | cardanol C15:1 | 519 | 4.8 | 2295 | 18.4 |
| | cardol C15:3 | 1567 | 14.4 | 1555 | 12.4 |
| | cardol C15:2 | 495 | 4.6 | 479 | 3.8 |
| | cardol C15:1 | 375 | 3.4 | 0 | 0.0 |
| | Total | 10878 | 100.0 | 12488 | 100.0 |

### Example 4

### Inhibition of Decarboxylation Reaction in Cases of Addition of Various Concentrations of Sulfuric Acids (pKa=−3)

10 g of a non-heated cashew nut shell liquid was taken in each beaker, and 0.1 wt %, 0.3 wt %, 0.6 wt %, and 1 wt % sulfuric acids were added thereto, respectively, followed by stirring to homogenize each mixture.

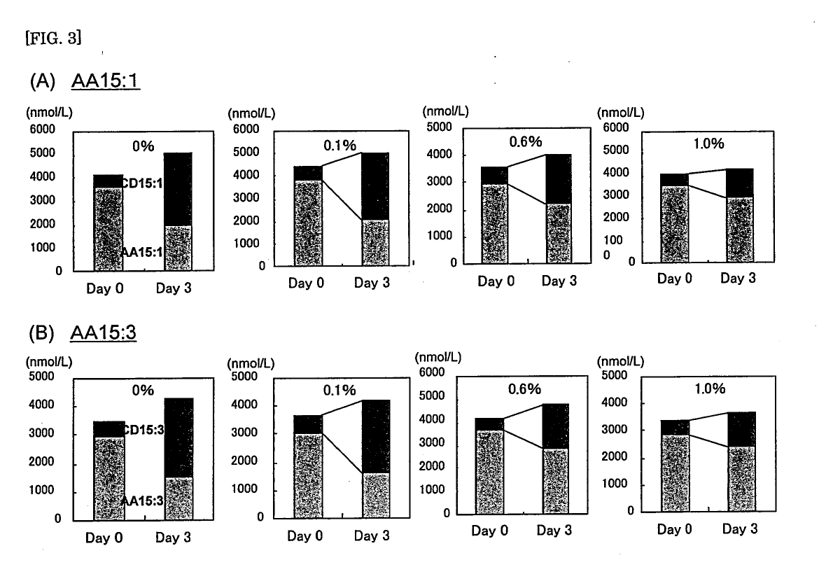

The respective beakers were sealed and then left to stand in a thermostat chamber at 80° C., and 3 days later, samples were collected and subjected to composition analyses by HPLC. The analysis results are separately shown for the anacardic acid 15:1 and the anacardic acid 15:3 as the concentrations in the analyses (nmol/L) (Table 3, left and right columns and FIGS. 3A and 3B). As is clear from the figure, the decarboxylation reaction decreased when a sulfuric acid was added in an amount of 0.6 wt %, and the decarboxylation reaction significantly decreased when a sulfuric acid was added in an amount of 1 wt %.

TABLE 3

| | |
|---|---|
| Day 0 | Day 3 |

Control

| | | | |
|---|---|---|---|
| C15:3 | AA | 2938 | 1543 |
| | CD | 588 | 2722 |
| C15:1 | AA | 3604 | 1978 |
| | CD | 564 | 3064 |

0.1% sulfuric acid

| | | | |
|---|---|---|---|
| C15:3 | AA | 3006 | 1614 |
| | CD | 612 | 2571 |
| C15:1 | AA | 3788 | 2074 |
| | CD | 603 | 2896 |

0.6% sulfuric acid

| | | | |
|---|---|---|---|
| C15:3 | AA | 2973 | 1745 |
| | CD | 606 | 2244 |
| C15:1 | AA | 3648 | 2264 |
| | CD | 568 | 2562 |

1.0% sulfuric acid

| | | | |
|---|---|---|---|
| C15:3 | AA | 2858 | 2426 |
| | CD | 570 | 1213 |
| C15:1 | AA | 3505 | 2988 |
| | CD | 546 | 1287 |

### Example 5

### Inhibition of Decarboxylation Reaction in Non-Heated Cashew Nut Shell Liquid Preparation (Preliminary Addition of Phosphoric Acid to Non-Heated Cashew Nut Shell Liquid)

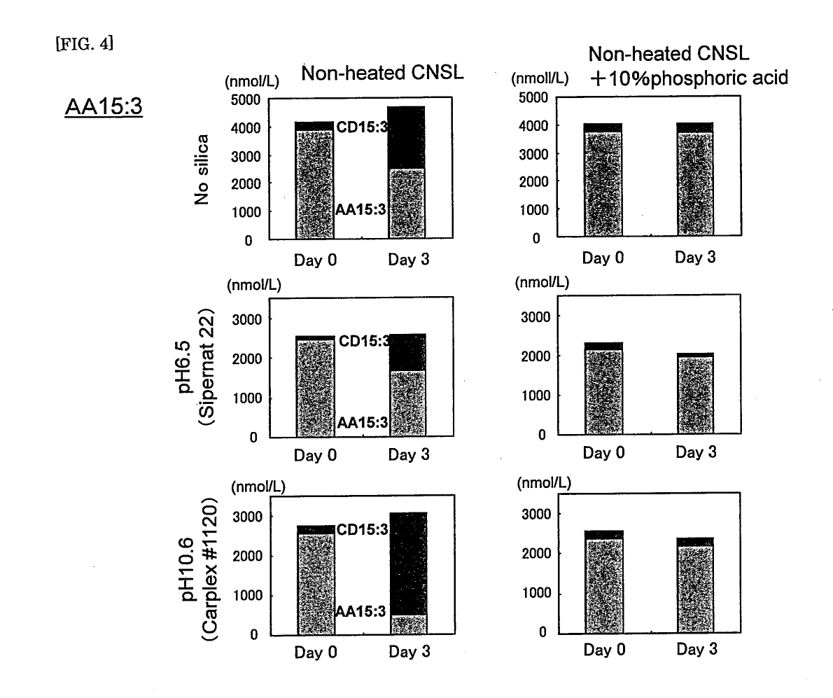

10 g of silica (Sipernat 22, Carplex #1120) were taken in each beaker, and 20 g of a non-heated cashew nut shell liquid or 20 g of a non-heated cashew nut shell liquid containing a 10 wt % phosphoric acid were added thereto, followed by stirring and mixing to homogenize each mixture. The respective beakers were sealed and then left to stand in a thermostat chamber at 80° C., and 3 days later, samples were collected and subjected to composition analyses by HPLC. The HPLC analyses were performed by: adjusting the concentrations of the collected samples with ethyl acetate so as to be 5 mg (including silica)/ml; then centrifuging the samples under conditions of 15,000 rpm and 5 min; and performing measurement for the resultant supernatants. The analysis results are shown as the concentrations (nmol/L) of the anacardic acid 15:3 in the analyses (Table 4 and FIG. 4).

In the case where silica, i.e., Sipernat 22 having an about neutral pH, was used as the carrier, the decarboxylation ratio was almost the same as or lower than that in the case of using no carrier, while in the case where an alkaline Carplex #1120 was used as the carrier, the decarboxylation ratio was found to apparently increase.

On the other hand, in the case where non-heated cashew nut shell liquid containing a 10 wt % phosphoric acid was used, the decarboxylation reaction was found to be inhibited regardless of the use of the carrier and of the pH of silica used as the carrier. The results are shown Table 4 below.

TABLE 4

| | |
|---|---|
| Day 0 | Day 3 |

Control

| | | | |
|---|---|---|---|
| C15:3 | Anacardic acid | 3852 | 2518 |
| | Cardanol | 315 | 2170 |

10% phosphoric acid

| | | | |
|---|---|---|---|
| C15:3 | Anacardic acid | 3757 | 3724 |
| | Cardanol | 279 | 310 |

S22/Control

| | | | |
|---|---|---|---|
| C15:3 | Anacardic acid | 2444 | 1664 |
| | Cardanol | 84 | 910 |

S22/10% phosphoric acid

| | | | |
|---|---|---|---|
| C15:3 | Anacardic acid | 2136 | 1936 |
| | Cardanol | 170 | 90 |

1120/Control

| | | | |
|---|---|---|---|
| C15:3 | Anacardic acid | 2548 | 512 |
| | Cardanol | 210 | 2530 |

1120/10% phosphoric acid

| | | | |
|---|---|---|---|
| C15:3 | Anacardic acid | 2375 | 2168 |
| | Cardanol | 190 | 205 |

### Example 6

### Inhibition of Decarboxylation Reaction in Non-Heated Cashew Nut Shell Liquid Preparation (Addition of Phosphoric Acid to Carrier in Advance)

150 g of a Sipernat 22 were collected in a plastic bag, and 9 ml of an 85% phosphoric acid was charged in a vaporizer and sprayed thereto while stirring. The pH of the Sipernat 22 supplemented with the phosphoric acid thus prepared was found to be 2.0 by measurement.

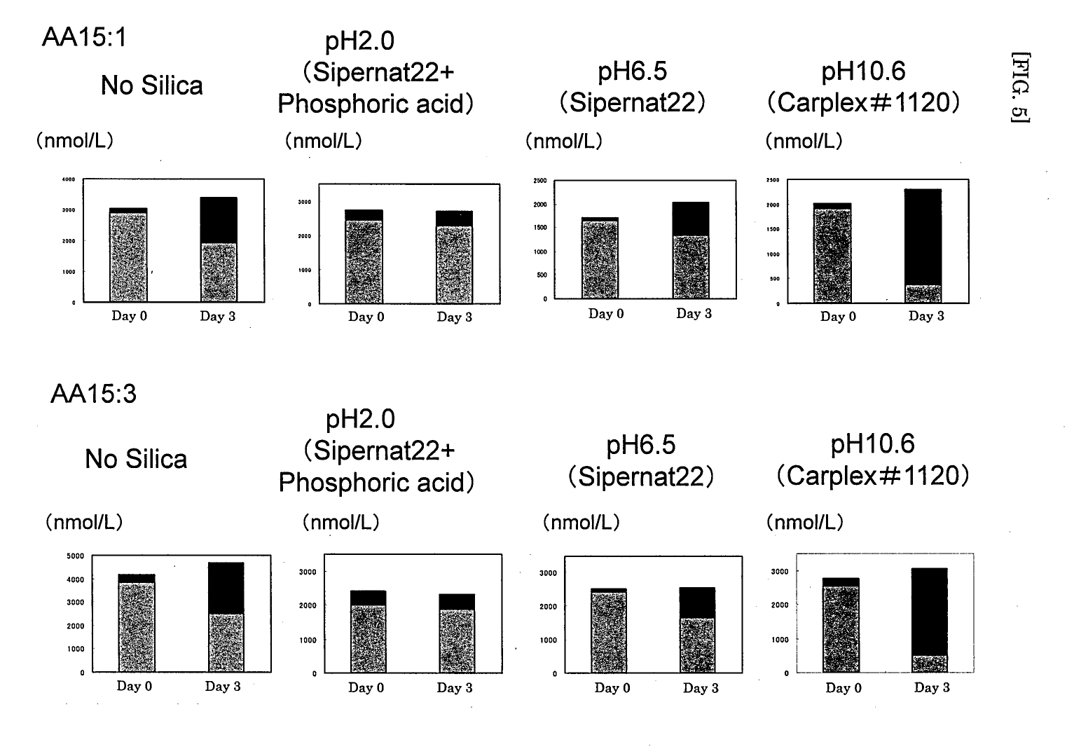

10 g of silica (Sipernat 22 supplemented with phosphoric acid, Sipernat 22, and Carplex #1120) were taken in each beaker, and 20 g of a non-heated cashew nut shell liquid were added thereto, respectively, followed by stirring and mixing to homogenize each mixture. The respective beakers were sealed and then left to stand in a thermostat chamber at 80° C., and 3 days later, samples were collected and subjected to composition analyses by HPLC. The HPLC analyses were performed by: adjusting the concentrations of the collected samples with ethyl acetate so as to be 5 mg (including silica)/ml; then centrifuging the samples under conditions of 15,000 rpm and 5 min; and performing measurement for the resultant supernatants. The analysis results are shown as the concentrations (nmol/L) of the anacardic acid 15:1 and the anacardic acid 15:3 in the analyses (Table 5 and FIG. 5).

The results are shown Table 5 below.

TABLE 5

| | |
|---|---|
| Day 0 | Day 3 |

AA15:1
No silica

| | | | |
|---|---|---|---|
| C15:1 | Anacardic acid | 2893 | 1919 |
| | Cardanol | 148 | 1473 |

pH 2.0(S22/phosphoric acid)

| | | | |
|---|---|---|---|
| C15:1 | Anacardic acid | 2450 | 2271 |
| | Cardanol | 295 | 420 |

pH 6.5/S22

| | | | |
|---|---|---|---|
| C15:1 | Anacardic acid | 1641 | 1354 |
| | Cardanol | 83 | 688 |

pH 10.6/1120

| | | | |
|---|---|---|---|
| C15:1 | Anacardic acid | 1902 | 387 |
| | Cardanol | 102 | 1912 |

AA15:3
No silica

| | | | |
|---|---|---|---|
| C15:3 | Anacardic acid | 3852 | 2518 |
| | Cardanol | 315 | 2170 |

pH 2.0(S22/phosphoric acid)

| | | | |
|---|---|---|---|
| C15:3 | Anacardic acid | 1998 | 1890 |
| | Cardanol | 409 | 440 |

pH 6.5/S22

| | | | |
|---|---|---|---|
| C15:3 | Anacardic acid | 2444 | 1664 |
| | Cardanol | 84 | 910 |

pH 10.6/1120

| | | | |
|---|---|---|---|
| C15:3 | Anacardic acid | 2548 | 512 |
| | Cardanol | 210 | 2530 |

The results reveal that the decarboxylation reaction is more inhibited as the pH of the carrier (silica) is lower.

In view of the foregoing, it is found that the acids to be used for the decarboxylation reaction are not necessary to be preliminarily added to the non-heated cashew nut shell liquids, and the acids may be added to the carriers in advance.

### Example 7

### Inhibition of Decarboxylation Reaction in Feed

To 100 g of a feed (standard feed for breeding young cattle: SDC No. 2 Nippon Formula Feed Mfg Co., Ltd.) were added 1 wt % two kinds of the silica preparations produced in Example 5 (10 g of Sipernat 22+20 g of non-heated cashew nut shell liquid or 20 g of non-heated cashew nut shell liquid containing a 10 wt % phosphoric acid), to thereby produce feeds each containing the non-heated cashew nut shell liquid.

The respective resultants were sealed and then left to stand in a thermostat chamber at 80° C., and 3 days later, samples were collected and subjected to composition analyses by HPLC.

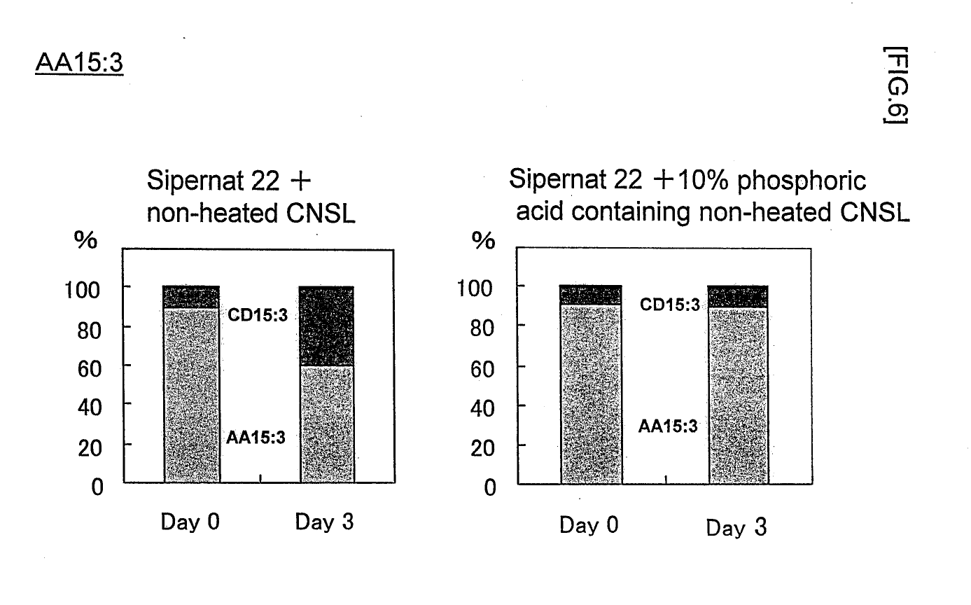

The HPLC analyses were performed by: extracting soluble parts with ethyl acetate from the feeds used in the test; filtrating and drying the extracts; adjusting the concentrations so as to be 5 mg/ml; and performing measurement. The analysis results show the compositions of the anacardic acid and the cardanol before and after the samples were left to stand in the thermostat chamber as for the anacardic acid 15:3 (Table 6 and FIG. 6).

The results are shown in Table 6 below.

TABLE 6

| | | Day 0 | | | | Day 3 | | | |
|---|---|---|---|---|---|---|---|---|---|
| | | Anacardic | | Cardanol | | Anacardic | | Cardanol | |
| | | acid 15:3 | | 15:3 | | acid 15:3 | | 15:3 | |
| Sipernat22 + | area | 16232 | | 1752 | | 9456 | | 5903 | |
| Non-heated CNSL | ppm | 386 | | 37 | | 225 | | 123 | |
| | n mol | 1129 | 90% | 123 | 10% | 658 | 61% | 414 | 39% |
| Sipernat22 + | area | 14356 | | 1489 | | 13389 | | 1411 | |
| 10% phosphoric | ppm | 341 | | 31 | | 318 | | 29 | |
| acid containing | n mol | 998 | 91% | 104 | 9% | 931 | 90% | 99 | 10% |
| Non-heated CNSL | | | | | | | | | |

This suggests that, in the case of using the non-heated cashew nut shell liquid containing a 10 wt % phosphoric acid, the anacardic acid is present stably without the progress of the decarboxylation in the feed.

### Example 8

### Production of Feed Supplemented with Non-Heated Cashew Nut Shell Liquid (Use of Organic Carrier)

A standard feed was used as a carrier to produce feeds for chicken and cattle. The formulations are not limited to the following examples.

### (1) Production of Feed for Broiler

| | |
|---|---|
| Standard feed for broiler in early fattening phase: | 99.5 wt % |
| SDB No. 1 Nippon Formula Feed Mfg Co., Ltd. | |
| Non-heated cashew nut shell liquid (10 wt % | 0.05 wt % |
| phosphoric acid) | |

When the components are mixed, a powder is obtained, and the solidification at about 20° C. can be prevented.

### (2) Production of Feed for Cattle

| | |
|---|---|
| Standard feed for breeding young cattle: SDC | 99.5 wt % |
| No. 2 Nippon Formula Feed Mfg Co., Ltd. | |
| Non-heated cashew nut shell liquid (10 wt % | 0.05 wt % |
| phosphoric acid) | |

When the components are mixed, a powder is obtained, and the solidification at about 20° C. can be prevented.

### INDUSTRIAL APPLICABILITY

The addition of an acid to a non-heated cashew nut shell liquid can inhibit foam formation due to decarboxylation. Further, the blending of an inorganic carrier in the non-heated cashew nut shell liquid supplemented with the acid can prevent the solidification at about 20° C. Therefore, the non-heated cashew nut shell liquid can be easily used for industrial applications.

1. A composition comprising an acid and a non-heated cashew nut shell liquid.

2. The composition according to claim 1, wherein the acid has a pKa of 4.5 or less.

3. The composition according to claim 1, wherein an amount of the acid is 0.5 wt % or more with respect to the non-heated cashew nut shell liquid.

4. A non-heated cashew nut shell liquid preparation, comprising the composition according to claim 1 and a carrier.

5. The non-heated cashew nut shell liquid preparation according to claim 4, wherein the carrier is silica.

6. A feed comprising a feed component and a composition according to claim 1.

7. A method of inhibiting a decarboxylation reaction of an anacardic acid in a non-heated cashew nut shell liquid, comprising adding an acid to the non-heated cashew nut shell liquid.

8. A method of producing a non-heated cashew nut shell liquid preparation according to claim 4, comprising mixing the acid with the carrier and further mixing the carrier mixed with the acid with the non-heated cashew nut shell liquid.

9. A method of inhibiting a decarboxylation reaction of an anacardic acid, comprising adding an acid to the anacardic acid.

10. A feed comprising a feed component and a non-heated cashew nut shell liquid preparation according to claim 4.

11. The composition according to claim 1, wherein said acid is present in said composition in such an amount that decarboxylation of an anacardic acid in the non-heated cashew nut is inhibited by the acid.

12. The composition according to claim 1, wherein said composition further comprises another acid.

13. A composition comprising an acid and anacardic acid.

14. The composition according to claim 13, wherein said acid is present in said composition in such an amount that decarboxylation of the anacardic acid is inhibited by the acid.

15. The composition according to claim 13, wherein the acid has a pKa of 4.5 or less.

16. The composition according to claim 13, wherein an amount of the acid is 0.5 wt % or more with respect to the anacardic acid.

17. The composition according to claim 13, wherein said composition further comprises another acid.

18. A anacardic acid preparation, comprising the composition according to claim 13 and a carrier.

19. The anacardic acid preparation according to claim 18, wherein the carrier is silica.

20. A feed comprising a feed component and the composition according to claim 13.

21. A feed comprising a feed component and the anacardic acid preparation according to claim 18.
